(19) United States
(12) Patent Application Publication
(10) Pub. No.: US 2020/0258640 A1
(43) Pub. Date: Aug. 13, 2020

(54) Remote medical diagnostic method and device therefor

(71) Applicant: SEOUL NATIONAL UNIVERSITY R&DB FOUNDATION, Seoul (KR)

(72) Inventors: Jongho LEE, Seoul (KR); Seung Hong Choi, Seoul (KR)

(21) Appl. No.: 16/271,910

(22) Filed: Feb. 11, 2019

(51) Int. Cl. G16H 80/00; G16H 50/20

(52) U.S. Cl. CPC G16H 80/00; G16H 50/20

(57) ABSTRACT

A method for a remote medical diagnostic service is disclosed. The method comprises a step of receiving from a terminal, by a service device, a readout result of readout request information including a part or all of medical information and reference medical information, a step of extracting, by the service device, a readout result for expert evaluation, which is content related to the reference medical information, out of the readout result, a step of comparing, by the service device, the readout result for expert evaluation with a reference readout result which is obtained in advance by reading out the reference medical information, and a step of calculating, by the service device, a service level of the medical information readout result, which is content related to the medical information, out of the readout result, based on the comparison result.

### BACKGROUND

The present invention relates to a technique for enabling remote medical diagnosis using a communication network device and a computing device.

Technological and commercial constraints for an implementation of remote medical diagnostics are disappearing due to changes in the medical environment, such as advances in medical diagnostic technology, changes in the supply and demand of medical experts, changes in the legal system of medical care, and development of communication technologies.

For example, a particular medical institution may directly hire a read-out expert to read out an MRI image measured from a patient. Alternatively, the medical institution may request reading of the MRI image from an external read-out expert not belonging to the medical institution.

Other types of medical information, as well as the MRI image, may also be subject to remote medical diagnosis, as long as it is information about the patient requesting a reading by a medical read-out expert.

However, if the expertise of an external read-out expert cannot be assessed, and it cannot be determined in real time whether an external read-out expert is in a state that is able to faithfully carry out a reading request from a medical institution, the decision of the medical institution about whether to utilize an external read-out expert will be withheld.

Therefore, the development of an evaluation system for external read-out expert will have a great impact on the introduction and proliferation of remote medical diagnostic technology.

### SUMMARY

One purpose of the present invention is to provide a technology for relaying information for a medical information readout service occurring between a medical diagnostic service customer such as a medical institution and an external read-out expert.

The present invention intends to provide basic information for evaluating the expertise of an external read-out expert to a medical diagnostic service customer and to provide a technique for real-time evaluation of the quality of service provided by an external read-out expert.

According to the present invention, a technology for relaying information for a medical information readout service occurring between a medical diagnostic service customer and an external read-out expert can be provided.

According to the present invention, it is possible to provide the basic information for the evaluation of the professional competence of the external read-out expert to the medical diagnostic service customer such as a medical institution, and to provide the technology for real-time evaluation of the service providing quality and/or the quality of service of the external read-out expert.

According to one aspect of the present invention, a method for a remote medical diagnostic service may be provided. The method comprises: a step of receiving, by a service device, a readout result of readout request information including a part or all of medical information and reference medical information from a terminal; a step of extracting, by the service device, a readout result for expert evaluation, which is content related to the reference medical information, out of the readout result; a step of comparing, by the service device, the readout result for expert evaluation with reference readout result which is obtained by reading out the reference medical information; and a step of calculating, by the service device, a service level of the medical information readout result, which is content related to the medical information, out of the readout result, based on the comparison result.

Here, the method may further comprise a step of transmitting to a requesting device information indicating that the calculated service level is less than or equal to a predetermined first value when the service device determines that the calculated service level is less than or equal to the predetermined first value.

Here, the method may further comprise a step of updating, by the service device, a rating for a read-out expert who has sent the read-out result on the basis of the calculated service level; a step of selecting, by the service device, one of a plurality of read-out experts including the read-out expert who has sent the read-out result, based on ratings for the plurality of read-out experts when the service device receives a remote diagnostic service request from a requesting device; and a step of transmitting, by the service device, information about the selected read-out expert to the requesting device.

Here, the service device may be configured to access a read-out expert database comprising a plurality of records having a first field representing a read-out expert and a second field representing a rating of corresponding read-out expert.

Here, the method may further comprises: before the step of receiving, a first step of receiving, by the service device, the medical information from a requesting device; before the step of receiving, a second step of transmitting, by the service device, the readout request information to the terminal; and a third step of transmitting, by the service device, the medical information readout result, to the requesting device.

Here, the service device may be capable of obtaining a first list of a plurality of read-out experts and a second list of a plurality of terminals or a plurality of contact account used by the plurality of read-out experts, and the terminal may be a terminal used by a read-out expert selected by a predetermined rule among the plurality of read-out experts.

Here, when the calculated service level is determined to be less than or equal to a predetermined value, the service device may be adapted to perform at least one of following steps: i) changing a setting of the service device so that a possibility of selecting a read-out expert who has sent the readout result is reduced, ii) changing the setting of the service device so that the read-out expert who has sent the readout result is excluded from a reading request for other medical information for a predetermined period of time, and iii) discarding the medical information readout result, and when the calculated service level is determined to be equal or higher than a predetermined second value, the service device may be adapted to perform changing a setting of the service device so that a possibility of selecting a read-out expert who has sent the readout result is increased.

Here, the step of calculating the service level may comprise: a step of calculating a third value for the service level based on a difference value between a first value indicated by a quantitative analysis result included in the reference readout result and a second value indicated by a quantitative analysis result included in the readout result for expert evaluation, or based on a ratio between the first value and the second value.

Here, the medical information may include at least one of image information detected from a living organism or a dead organism and acoustic information detected from a living organism or from a dead organism, and the medical information may be provided for diagnosing a condition of a living organism or provided for diagnosing a condition of a dead organism.

Here, the medical information may be medical information on one patient or medical information on a plurality of patients, the requesting device may be a server managed by a medical diagnosis service consumer, the service device may relay information exchange between the requesting device and the terminal, and the terminal may be a computing device used by a read-out expert having ability to read the medical information.

Here, information that can identify a patient's identity may not be included in the medical information, or the medical information may include information that can specify a patient's identity but information that can specify the patient's identity may not be included in the readout request information.

Here, the method may further comprise: a step of receiving, by the service device, an evaluation score regarding the medical information readout result from the requesting device or another device, or acquiring, by the service device, an evaluation score through a user interface of the service device; and a step of determining, by the service device, a rating of the read-out expert who has read the medical information based on at least one of the evaluation score and the service level and updating a compensation level for the read-out expert based on the determined rating. Here, the service device may be adapted to include in the readout request information identification information identifying a read-out expert who will read the medical information and the readout result may be deemed to be derived by a read-out expert identified by the identification information.

Alternatively, the method may further comprise: before the step of receiving, by the service device, a step for receiving a remote diagnosis service request from a requesting device; and before the step of receiving, by the service device, a step for selecting a read-out expert, and providing the requesting device with contact information of the selected read-out expert. Here, the readout result may be received from the terminal not passing though the requesting device or passing through the requesting device, and the readout request information may be transferred from the requesting device to the terminal without passing through the service device.

Here, the method may further comprise: before the step of receiving, by the service device, a step for providing the requesting device with the reference medical information; or before the step of receiving, by the service device, a step for acquiring the reference readout result from the requesting device.

According to another aspect of the present invention, a service device comprising a communication unit, and a processing unit may be provided. Here, the processing unit is configured to: using the communication unit, receive a readout result of readout request information including a part or all of medical information and reference medical information from a terminal; extract a readout result for expert evaluation, which is content related to the reference medical information, out of the readout result; compare the readout result for expert evaluation with reference readout result which is obtained by reading out the reference medical information; and calculate a service level of the medical information readout result, which is content related to the medical information, out of the readout result, based on the comparison result.

Here, the processing unit may be further configured to: before receiving the readout result, using the communication unit, receive the medical information from a requesting device; before receiving the readout result, using the communication unit, transmit the readout request information to the terminal; and using the communication unit, transmit the medical information readout result, to the requesting device.

According to still another aspect of the present invention, a non-transitory computer readable storage medium storing one or more programs can be provided. Here, the one or more programs comprises instructions, which when executed by an electronic device, cause the electronic device to: receive a readout result of readout request information including a part or all of medical information and reference medical information from a terminal; extract a readout result for expert evaluation, which is content related to the reference medical information, out of the readout result; compare the readout result for expert evaluation with reference readout result which is obtained by reading out the reference medical information; and calculate a service level of the medical information readout result, which is content related to the medical information, out of the readout result, based on the comparison result.

Here, when the instructions are executed by the electronic device, the instructions may further cause the electronic device to: before receiving the readout result, receive the medical information from a requesting device; before receiving the readout result, transmit the readout request information to the terminal; and transmit the medical information readout result, to the requesting device.

### DESCRIPTION OF THE FIGURES

FIG. 1 is a flow chart for explaining a remote medical diagnosis method according to an embodiment of the present invention.

FIG. 2 is a flowchart illustrating a method for selecting a specific read-out expert according to an embodiment of the present invention.

FIG. 3 is a flowchart illustrating a remote medical diagnosis method according to an embodiment of the present invention.

FIG. 4 shows an example of a configuration of a remote medical diagnosis system for executing a remote medical diagnostic method provided in accordance with an embodiment of the present invention.

FIG. 5 is a flow diagram illustrating a method of remote medical diagnostic service provided in accordance with an embodiment of the present invention.

FIG. 6 is a flow chart illustrating a method of remote medical diagnostic service provided in accordance with another embodiment of the present invention.

FIG. 7 is a flowchart illustrating a method of utilizing the calculated service level according to an embodiment of the present invention.

FIG. 8 is a flowchart illustrating a method of utilizing the calculated service level according to another embodiment of the present invention.

### DETAILED DESCRIPTION

Exemplary embodiments in accordance with principles of inventive concepts will now be described more fully with reference to the accompanying drawings, in which exemplary embodiments are shown. Exemplary embodiments in accordance with principles of inventive concepts may, however, be embodied in many different forms and should not be construed as being limited to the embodiments set forth herein; rather, these embodiments are provided so that this disclosure will be thorough and complete, and will fully convey the concept of exemplary embodiments to those of ordinary skill in the art. Like reference numerals in the drawings denote like elements, and thus their description may not be repeated. Like numbers indicate like elements throughout. As used herein the term “and/or” includes any and all combinations of one or more of the associated listed items.

It will be understood that, although the terms “first”, “second”, etc. may be used herein to describe various elements or components, these elements or components should not be limited by these terms. These terms are only used to distinguish one element or component from another element or component. Thus, a first element or component discussed below could be termed a second element or component without departing from the teachings of exemplary embodiments.

The terminology used herein is for the purpose of describing particular embodiments only and is not intended to be limiting of exemplary embodiments. As used herein, the singular forms “a,” “an” and “the” are intended to include the plural forms as well, unless the context clearly indicates otherwise. It will be further understood that the terms “comprises”, “comprising”, “includes” and/or “including,” if used herein, specify the presence of stated features, integers, steps, operations, elements and/or components, but do not preclude the presence or addition of one or more other features, integers, steps, operations, elements, components and/or groups thereof.

Exemplary embodiments in accordance with principles of inventive Unless otherwise defined, all terms (including technical and scientific terms) used herein have the same meaning as commonly understood by one of ordinary skill in the art to which exemplary embodiments in accordance with principles of inventive concepts belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

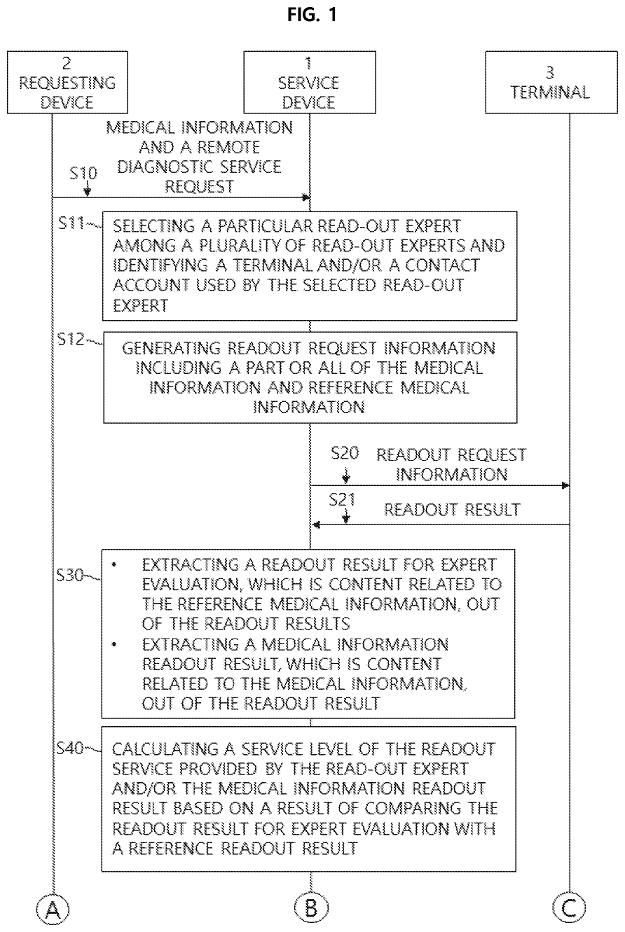

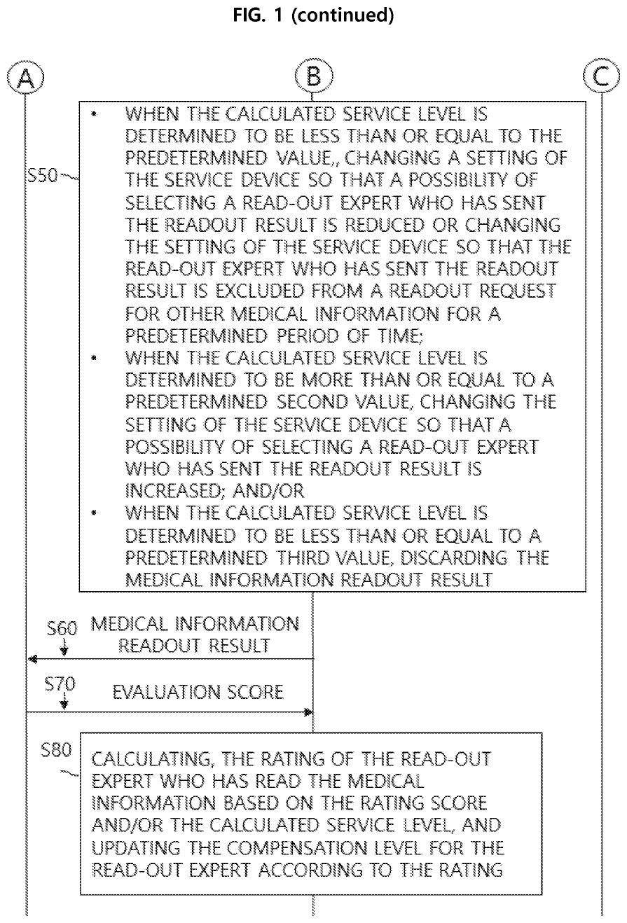

Hereinafter, with reference to FIG. 1, a remote medical diagnosis method provided in accordance with an embodiment of the present invention will be described.

FIG. 1 is a flow chart for explaining a remote medical diagnosis method according to an embodiment of the present invention.

In this specification, the term ‘remote medical’ may include the concept of ‘remote medical treatment’. As used herein, the term ‘medical’ may include medical actions for the treatment of humans or animals, as well as acts for the purpose of studying living or dead organisms.

The remote medical diagnosis method according to an embodiment of the present invention may include step S10, step S11, step S12, step S20, step S21, step S30, step S40, step S50, step S60, step S70, and step S80 as described below.

Step S10: a step of receiving, by a service device 1, medical information and a remote diagnostic service request from a requesting device 2;

Step S11: a step of selecting, by the service device 1, a particular read-out expert among a plurality of read-out experts and identifying a terminal 3 and/or a contact account used by the selected read-out expert;

Step S12: a step of generating, by the service device 1, readout request information including a part or all of the medical information and reference medical information stored in advance;

Step S20: a step of transmitting, by the service device 1, the readout request information to the terminal 3 used by the finally selected read-out expert;

Step S21: a step of receiving, by the service device 1, a readout result for the readout request information from the terminal 3;

Step S30: a step of extracting, by the service device 1, a readout result for expert evaluation, which is content related to the reference medical information, out of the readout results, and extracting a medical information readout result, which is content related to the medical information, out of the readout result;

Step S40: a step of calculating, by the service device 1, a service level of the readout service provided by the read-out expert and/or the medical information readout result based on a result of comparing the readout result for expert evaluation with a reference readout result;

Step S50: when the calculated service level is determined to be less than or equal to the predetermined value, by the service device 1, changing a setting of the service device so that a possibility of selecting a read-out expert who has sent the readout result is reduced or changing the setting of the service device so that the read-out expert who has sent the readout result is excluded from a readout request for other medical information for a predetermined period of time; when the calculated service level is determined to be more than or equal to a predetermined second value, by the service device 1, changing the setting of the service device so that a possibility of selecting a read-out expert who has sent the readout result is increased; and/or when the calculated service level is determined to be less than or equal to a predetermined third value, discarding the medical information readout result;

Step S60: a step of transmitting, by the service device 1, a medical information readout result, which is content related to the medical information, out of the readout result to the requesting device 2, if the medical information readout result has not been discarded;

Step S70: a step of receiving, by the service device 1, an evaluation score regarding the medical information readout result from the requesting device or another device or having an input of the evaluation score from a user interface of the service device 1; and

Step S80: a step of calculating, by the service device 1, the rating of the read-out expert who has read the medical information based on the rating score and/or the calculated service level, and updating the compensation level for the read-out expert according to the rating.

The steps described above are described in more detail below.

### Detailed Description of Step S10

The medical information presented in step S10 may include measurement data that is data measured from a patient's body as information for which reading by a read-out expert is required.

In one embodiment, the measurement data may include one or more of image, moving pictures, and audio. However, the measurement data is not limited to image, moving pictures, and audio, and may be data having attributes different from those of image, moving pictures, and audio. For example, the measurement data may be an MRI image or a CT image or an ultrasound image of a body of a patient A.

In addition, the medical information may optionally include history information of a patient. That is, in order for the measurement data to be accurately read, there may be a case where a patient's past medical history is required.

In addition, the medical information may optionally include physical information such as sex, age, height, weight, and blood type of the patient.

In addition, the medical information may optionally include personal information for identifying the patient from another person.

The medical information may be medical information relating to one or more patients. That is, the requesting device may request readout of medical information for one patient, but may request readout of medical information for a plurality of patients at one time.

According to one embodiment of the present invention, information that can identify a patient's identity may not be included in the medical information, or the medical information includes information that can specify a patient's identity but information that can specify the patient's identity may not be included in the readout request information.

According to one embodiment of the present invention, the medical information may include at least one of image information detected from a living organism or a dead organism and acoustic information detected from a living organism or from a dead organism. And the medical information may be provided for diagnosing a condition of a living organism or provided for diagnosing a condition of a dead organism.

The requesting device presented in step S10 may be a server or a computing device managed by a medical institution performing face-to-face medical diagnosis for the one or more patients or by a medical research institution. Otherwise, the requesting device may be a server or a computing device managed by an arbitrary medical diagnosis service customer.

The service device 1 presented in step S10 may be connected to a plurality of the requesting devices via a communication network. The requesting device presented in step S10 may be any one of the plurality of requesting devices. The plurality of requesting devices may be different devices managed by different medical institutions or medical research institutions. Otherwise, each the plurality of requesting devices may be a server or a computing device managed by an arbitrary medical diagnosis service customer.

The service device 1 may be a computing device or a server including a processing unit and a communication unit. The service device 1 can access a communication network through the communication unit. The service device 1 can communicate with the requesting device 2 and the terminal 3 via the communication network.

The service device 1 may be adapted to obtain a read-out expert list which is a list of a plurality of read-out experts and a terminal list which is a list of a plurality of terminals used by the plurality of read-out experts from other device. Or, the service device 1 may be storing the read-out expert list and the terminal list.

In one embodiment, the requesting device 2 cannot directly connect to the terminal 3 and can connect to the terminal 3 only through the service device 1. In addition, in one embodiment, the terminal 3 cannot connect directly to the requesting device 2, but can only connect to the requesting device 2 via the service device 1.

However, in another embodiment, the requesting device 2 and the terminal 3 may be directly connected to each other. However, even in this case, the service device 1 can provide a predetermined service for relaying between the requesting device 2 and the terminal 3.

### Detailed Description of Step S11

In step S11, the service device 1 can select a specific read-out expert among the plurality of read-out experts included in the read-out expert list, and identify the terminal 3 used by the selected read-out expert. Alternatively, instead of directly identifying the terminal 3, it is possible to identify a contact account used by the selected read-out expert. Through the contact account, the selected read-out expert can access the data sent by the service device 1 to the selected read-out expert using an arbitrary terminal. The contact account may be, for example, an email address.

The communication between the service device 1, the requesting device 2, and the terminal 3 may be assisted by a third Internet service provider not shown in FIG. 1.

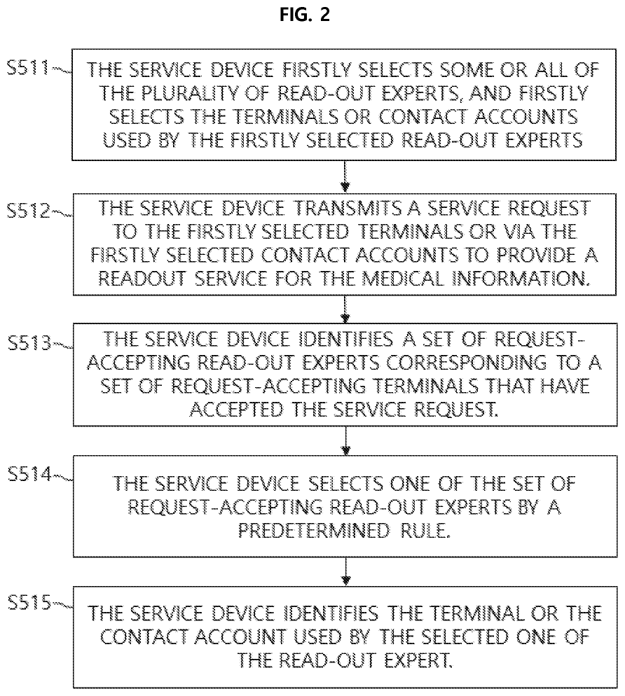

Hereinafter, a method for selecting a specific read-out expert according to an embodiment of the present invention will be described with reference to FIG. 2.

FIG. 2 is a flowchart illustrating a method for selecting a specific read-out expert according to an embodiment of the present invention.

To this end, in step S511, the service device 1 may firstly select some or all of the plurality of read-out experts, and may firstly select the terminals or contact accounts used by the firstly selected read-out experts.

Then, in step S512, the service device 1 may transmit a service request to the firstly selected terminals or via the firstly selected contact accounts to provide a readout service for the medical information.

The service request may include information about the type of medical information that each read-out expert should read, the amount of medical information that each read-out expert should read, and information about the cost of the readout service provided by each read-out expert have.

The quality of service or customer satisfaction that each read-out expert can provide may vary depending on the expertise of each read-out expert. Thus, each read-out expert can be rated based on the experience of each read-out expert or on the basis of the evaluation information about the readout services that each read-out expert provided in the past. The information about the cost of the readout service included in the service request may vary according to the rating of the read-out expert who is to receive the service request.

For example, a first service request for requesting a readout service for the first medical information may be sent to a first read-out expert and a second service request for requesting a readout service for the first medical information may be sent to a second read-out expert.

At this time, information on the type of the first medical information may be included in both the first service request and the second service request. For example, the information on the type of the first medical information may be information indicating that it is an MRI image of an adult brain.

The first service request and the second service request may include information on the amount of the first medical information, respectively. For example, both the first service request and the second service request may include information on the number of MRI images of the adult brain to be read by each read-out expert.

And the first service request may include a value of a first level that is a level of a reward to be provided to the first read-out expert as a compensation for providing a readout service for the first medical information. And the second service request may include a value of a second level that is a level of a reward to be provided to the second read-out expert as a compensation for providing a readout service for the first medical information. The first level and the second level may be different or equal to each other.

Each of the read-out experts may determine, based on the type of medical information to be read, the amount of medical information to be read, and the amount of reward that is to be provided a, he or she can decide whether or not to accept the service request.

In step S513, the service device 1 can then identify a set of request-accepting read-out experts corresponding to a set of request-accepting terminals that have accepted the service request.

In step S514, the service device 1 can then select one of the set of request-accepting read-out experts by a predetermined rule.

And then, in step S515, the service device 1 may identify the terminal 3 or the contact account used by the selected one of the read-out expert.

The process of firstly selecting some or all of the plurality of read-out experts and/or selecting one among the set of request-accepting read-out experts may include observing ratings assigned to the plurality of read-out experts, and selecting one or more read-out experts based on the assigned ratings.

For example, the process of firstly selecting some or all of the plurality of read-out experts may include filtering out read-out experts having a rating less than a predetermined level.

And the process of selecting, for example, one of the set of request-accepting read-out experts, may include selecting a read-out expert of the highest rating among the set of request-accepting read-out experts.

According to one embodiment of the present invention, the service device may relay information exchange between the requesting device and the terminal, and the terminal may be a computing device used by a read-out expert having ability to read the medical information.

### Detailed Description of Step S12

The readout request information presented in step S12 may include a part or all of the medical information. For example, when the medical information received by the service device 1 from the requesting device 2 includes personal information, the readout request information may include only a part of the medical information except for the personal information. Alternatively, for example, if the medical information received by the service device 1 from the requesting device 2 includes only an MRI image of the brain of an anonymous patient, the readout request information may include all of the medical information.

The reference medical information presented in step S12 and the reference readout result presented in step S40 may be information previously stored in the service device 1 or stored in advance in another device to which the service device 1 can have an access. At this time, the another device may be a data server that the service device 1 can access.

In one embodiment of the present invention, the reference medical information may be the same type of information as the medical information. For example, if the medical information is an MRI image of the brain of a patient A, the reference medical information may be an MRI image of the brain of a patient B. In another embodiment, the reference medical information may be a type of information similar to the medical information. For example, if the medical information is an MRI image of the brain of a patient A, the reference medical information may be an MRI image of the liver of a patient B. However, in another embodiment, the reference medical information may be other types of information than the medical information.

The reference readout result may be one that has been read out from the reference medical information beforehand and has been sufficiently verified by one or more read-out experts. That is, the reference readout result may be a readout result of the reference medical information that is perceived as correct.

According to one embodiment of the present invention, the service device may be adapted to include in the readout request information identification information identifying a read-out expert who will read the medical information.

### Detailed Description of Step S21 and Step S30

The readout result presented in step S21 and step S30 may include a ‘medical information readout result’ which is the result of reading the part or all of the medical information by the finally selected read-out expert. The readout result may include a ‘readout result for expert evaluation’ that is a result of reading the reference medical information.

The medical information readout result and the readout result for expert evaluation may be exemplified as follows to help understand the meaning of the medical information readout result and the readout result for expert evaluation.

That is, for example, when a part or the whole of the medical information is an MRI image of the brain of a first patient, the medical information readout result may include quantitative information on the presence or absence of a lesion, the number of lesions to be distinguished from each other, and the size of each lesion.

For example, when the reference medical information is an MRI image of a brain of a reference patient, the readout result for expert evaluation may include quantitative information on the presence or absence of a lesion, the number of lesions to be distinguished from each other, and the size of each lesion.

In addition, the medical information readout result and the readout result for expert evaluation may each include a qualitative analysis result such as a sentence described by analyzing a part or whole of the medical information and the reference medical information.

The medical information readout result and the readout result for expert evaluation may each include at least one of a quantitative analysis result and a qualitative analysis result.

According to one embodiment of the present invention, the readout result may be deemed to be derived by a read-out expert identified by the identification information.

### Detailed Description of Step S40

The step of calculating the service level represented in step S40 may comprise a step S410 of calculating a third value for the service level based on a difference value between a first value indicated by a quantitative analysis result included in the reference readout result and a second value indicated by a quantitative analysis result included in the readout result for expert evaluation, or on a ratio between the first value and the second value.

Alternatively, the step of calculating the service level may include the steps of: comparing the first sentence indicated in the qualitative analysis result included in the reference readout result with the content of the second sentence indicated by the qualitative analysis result included in the readout result for expert evaluation to calculate a degree of matching between the contents of the first sentence and the contents of the second sentence. The degree of matching can be calculated using artificial intelligence. The degree of matching is converted into a numerical value and can be regarded as a value relating to the service level.

The calculation of the service level relates to the quality and/or reliability of the medical information readout result, which is the result of reading the medical information sent by the requesting device 2, or the satisfaction of the consumer of the medical information readout result. Or the calculation of the service level is related to the overall quality and/or reliability of the readout result provided by the read-out expert through step S21, or the customer's satisfaction with the readout result.

One method of evaluating the service level of the medical information readout result, that is, the reliability of the medical information readout result, is a method in which another expert by himself directly verifies the medical information readout result for the medical information. However, in this case, since the another expert can be hired anyway, there is no need to request the finally selected read-out expert to read the medical information. Or, if both of the service of the finally selected read-out expert and the service of the other specialist are provided, it is very unreasonable in terms of cost expenditure because it results in double processing of work. Therefore, it is understandable that using this method is very unreasonable.

Yet another method of evaluating the service level of the medical information readout result, i.e., the reliability of the medical information readout result, is not to directly evaluate the reliability (service level) of the medical information readout result. As shown by the step S40 of one embodiment of the present invention, the reliability of the medical information readout result may be indirectly evaluated.

It is reasonable to assume that the medical information readout result and the readout result for expert evaluation would have been derived by the same person. It can also be assumed that the medical information readout result and the readout result for expert evaluation are derived at substantially similar time. Therefore, it is a reasonable choice to calculate the reliability of the readout result for expert evaluation and regard it as the reliability of the medical information readout result.

According to an embodiment of the present invention, the step of calculating the service level may be executed automatically in the service device 1. And, when the finally selected read-out expert returns the readout result through the terminal 3 to the service device 1, the step of calculating the service level can be executed immediately. Thus, it can be appreciated that the calculation of the service level can be made substantially in real time.

### Detailed Description of Step S50

If it is determined in step S50 that the calculated service level is less than or equal to the predetermined value, a penalty is imposed on the finally selected read-out expert that returned the readout result basically, or the reliability evaluation score for the readout result is lowered, or the readout result may be discarded. Further, the read-out expert may not be requested to provide the readout service for a predetermined period or more.

For example, if the calculated service level is below the determined third value, the medical information readout result may be discarded.

If the returned readout result returned from the finally selected read-out expert is discarded, the service device 1 can return to step S11 and select a new read-out expert. Then, the service device 1 can again execute the subsequent steps.

If the calculated service level is less than or equal to the predetermined value, a method of imposing a penalty on the finally selected read-out expert or on the readout result may be variously implemented.

On the other hand, when the calculated service level is equal to or higher than the determined second value, it is possible to increase the possibility of selecting the read-out expert who sent the readout result as a future remote diagnosis service provider.

Consumers of remote medical diagnostic services will be able to rely on the remote medical diagnostic platform using the service device 1 because the service device 1 provides a reliable technology that automatically excludes subordinate or low level read-out expert. Thus, the technique according to an embodiment of the present invention for determining the service level of the readout results provided by any read-out expert substantially in real time can be used as an underlying technology to activate the field of remote medical diagnosis.

### Detailed Description of Step S60, Step S70, and Step S80

Optionally, after step S60, the requesting device 2 may receive a user input for finally finalizing the received medical information readout result. If the user of the requesting device 2, that is, the consumer of the medical information readout result, refuses to finally finalize the received medical information readout result, the requesting device 2 sends such rejection notice to the service device 1. When the service device 1 receives the rejection notice, the service device 1 can request the terminal 3 to read out the readout request information again. Thereafter, steps S21 and subsequent steps may be repeated and executed again.

The evaluation score presented in step S70 may be calculated by a person or a medical institution using the requesting device 2 that has received the medical information readout result. Another expert who has the ability to read the medical information himself as a consumer of the medical information readout result may compare the medical information readout result with the medical information, and then decide a value of an indicator such as the accuracy or reliability of the medical information readout result. The value of the indicator can be fed back to the service device 1 with various routes.

The another expert using the requesting device 2 can read the medical information by himself/herself. However, according to various circumstances, the another expert can refer the reading of the medical information to an external read-out expert, that is, the finally selected read-out expert. If the time period consumed for reading the medical information is a first time period, and if the time period for verifying the medical information readout result already provided is a second time period, the second time period may be shorter than the first time period. In this case, it is understood that the another expert may wish to request the external read-out expert for reading rather than reading the medical information by himself/herself.

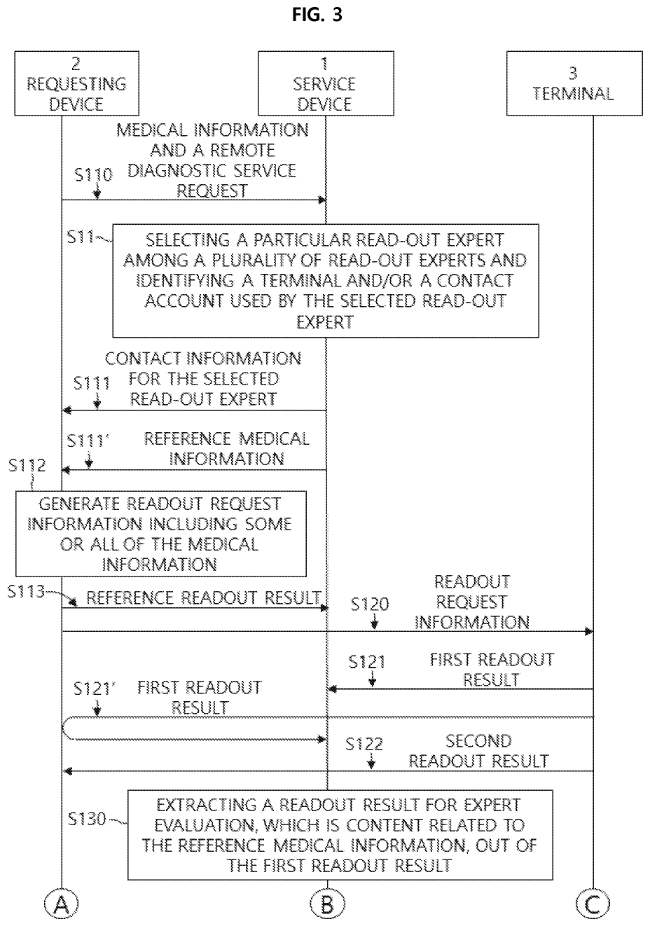

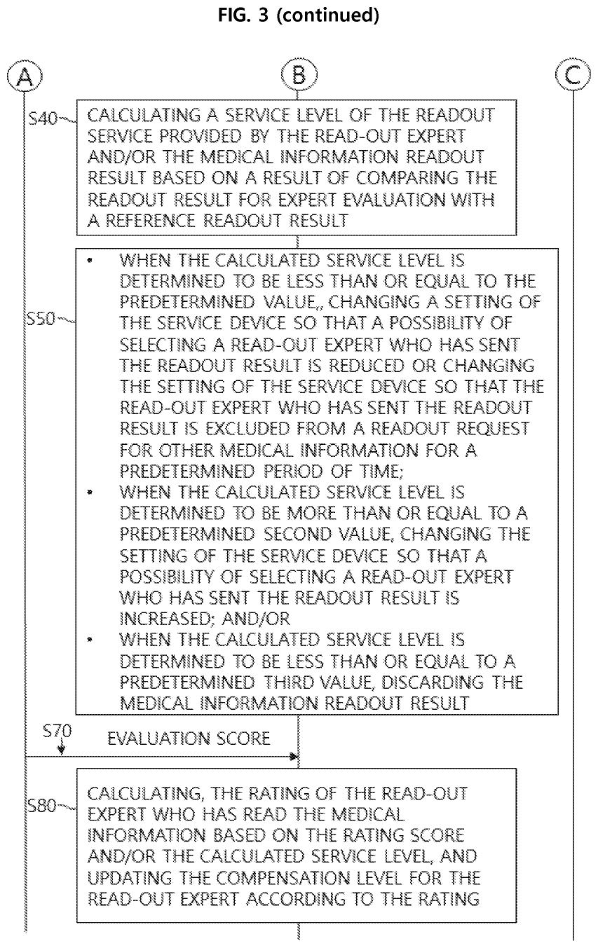

Hereinafter, a remote medical diagnosis method provided in accordance with another embodiment of the present invention will be described with reference to FIG. 3.

FIG. 3 is a flowchart illustrating a remote medical diagnosis method according to an embodiment of the present invention. FIG. 3 shows a modified method from FIG. 1.

In FIG. 1, the communication between the requesting device 2 and the terminal 3 is relayed by the service device 1. In contrast, in FIG. 3, the service device 1 performs only a part of procedures necessary for relaying the remote medical diagnosis service between the requesting device 2 and the terminal 3, and the other procedures may be performed by a direct communication between the requesting device 2 and the terminal 3.

The steps indicated by the same reference numerals in FIG. 1 and FIG. 3 are identical to each other. For convenience of explanation, only the difference between the method shown in FIG. 3 and the method shown in FIG. 1 will be described. Even if this is done, it is easy to understand the method presented in FIG. 3.

Step S40, step S50, step S60, step S70, and step S80 shown in FIG. 3 may be the same as those shown in FIG. 1.

The step S60 shown in FIG. 1 may be omitted in FIG. 3.

Step S10 shown in FIG. 1 may be replaced by step S110 in FIG. 3.

Step S12 shown in FIG. 1 may be replaced with step S112 in FIG. 3.

Step S20 shown in FIG. 1 may be replaced by step S120 in FIG. 3.

Step S21 shown in FIG. 1 may be replaced by step S121 in FIG. 3.

Step S30 shown in FIG. 1 may be replaced with step s130 in FIG. 3.

In FIG. 3, step S111 may be added between step S11 and step S112. In addition, in FIG. 3, step S111′ may optionally be added between step S11 and step S112.

In FIG. 3, step S113 may be added as an option.

In FIG. 3, step S122 may be added between step S120 and step S130. Also, in FIG. 3, step S121′ may optionally be added between step S120 and step S130.

In step S110. The service device 1 may receive medical information and a remote diagnosis service request from the requesting device 2. At this time, depending on the embodiment, the service device 1 may receive only the remote diagnosis service request from the requesting device 2 without the medical information. Except for these differences, other specific details to be executed in step S110 may be implemented in the same manner as step S10 in FIG. 1.

In step S111, the service device 1 can provide the requesting device 2 with the contact information on the read-out expert selected in the step 11. The contact information may be, for example, an identifier of a terminal used by the read-out expert, or a contact account used by the read-out expert.

In step S111′, the service device 1 can prepare the reference medical information described in FIG. 1 and provide the reference medical information to the requesting device 2. Depending on the embodiment, step S111′ may be omitted in FIG. 3.

In step S112, the requesting device 2 may generate readout request information including some or all of the medical information, and reference medical information prepared in advance. In FIG. 1, the readout request information is generated by the service device 1, but in FIG. 3, the requesting device 2 generates the readout request information.

In one embodiment, the previously prepared reference medical information may be obtained from the service device 1 by step S111′.

In another embodiment, the previously prepared reference medical information may be prepared by the requesting device 2 itself without the assistance of the service device 1. That is, the reference medical information and the reference readout result presented in step S40 of FIG. 1 may be information previously stored in the requesting device 2, or may be stored in advance in another device accessible to the requesting device 2. Where the another device may be a data server that the requesting device 2 can access.

Other specific details to be executed in step S112, except for the differences described above, may be implemented in the same manner as step S12 in FIG. 1.

If the reference medical information prepared in advance is prepared by the requesting device 2 itself without help of the service device 1, the requesting device 2 may provide the reference readout result to the service device 1 in step S113 for the use of the service device 1 in the subsequent step.

The requesting device 2 can directly transmit the readout request information to the terminal 3 in step S120. At this time, the readout request information may not pass through the service device 1. In step S120, the requesting device 2 may use the contact information acquired in step S111. Other specific details to be executed in step S120, except for this difference, may be implemented in the same manner as step S20 in FIG. 1.

In step S121, the terminal 3 can provide the service device 1 with a first readout result. The first readout result may include the readout result for expert evaluation presented in step S30 of FIG. 1. According to the embodiment, the first readout result may further include the medical information readout result presented in step S30 of FIG. 1, and in this case, the first readout result may be the same as the readout result presented in step S30 of FIG. 1.

In step S121′, the terminal 3 may provide the first readout result to the service device 1 via the requesting device 2. To this end, the requesting device 2 can provide the first readout result to the service device 1 automatically or by user input.

Only one of the step S121 and the step S121′ may be performed, or both of them may be performed in FIG. 3.

In step S122, the terminal 3 may provide the requesting device 2 with a second readout result. The second readout result may include the medical information readout result presented in step S30 of FIG. 1. According to the embodiment, the second readout result may further include the readout result for expert evaluation presented in step S30 of FIG. 1, and in this case, the second readout result may be the same as the readout result presented in step S30 of FIG. 1.

Optionally, after step S122, the requesting device 2 may receive a user input that finally confirms the received medical information readout result. If the user of the requesting device 2, that is, the consumer of the medical information readout result, refuses to finally finalize the received medical information readout result, the requesting device 2 transmits a rejection notice to the terminal 3 or service device 1. When the service device 1 receives the rejection notice, the service device 1 can request the terminal 3 to read out the readout request information again. When the terminal 3 receives this rejection notice, it can again request the readout expert to read out the readout request information. Thereafter, steps S121 and subsequent steps may be repeated and executed again.

In step S130, the service device 1 can extract the readout result for expert evaluation, which is the content related to the reference medical information, out of the first readout result. Other specific details to be executed in step S130, except for this difference, may be implemented in the same manner as step S30 of FIG. 1, if possible.

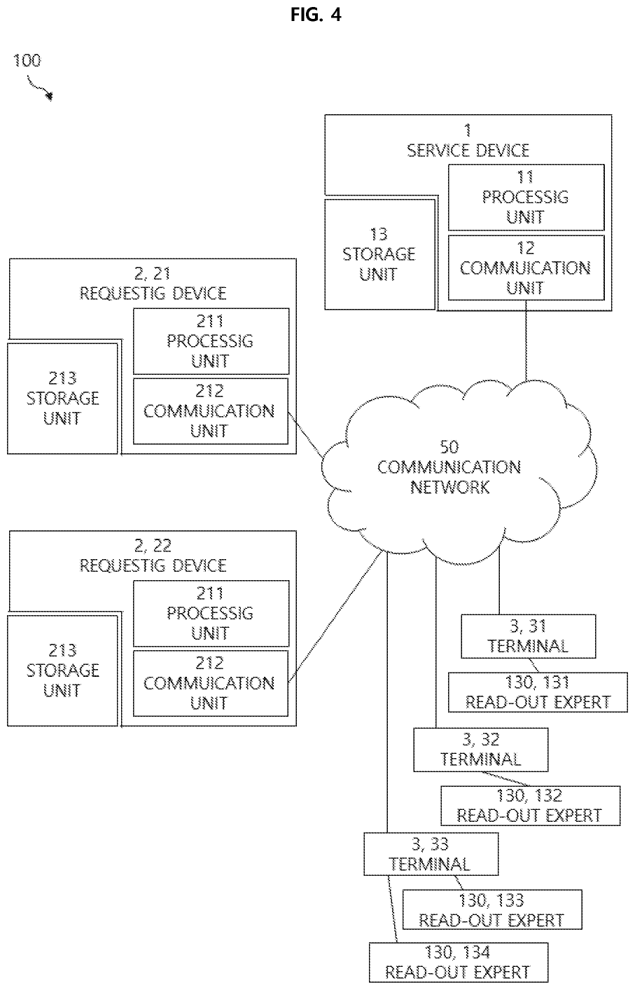

FIG. 4 shows an example of a configuration of a remote medical diagnosis system for executing a remote medical diagnostic method provided in accordance with an embodiment of the present invention.

The remote medical diagnosis system 100 may include a service device 1, one or more requesting devices 2, and one or more terminals 3. Said devices and terminals may include a communication electric circuit capable of communicating with each other via the communication network 50.

The remote medical diagnosis system 100 may include a plurality of requesting devices 2. Each requesting device 2 may freely choose to connect to and disconnect from the remote medical diagnostic system 100. Each requesting device 2 may be, for example, one used by different medical institutions or different medical research institutes, or by different medical practitioners or medical practitioners. That is, each requesting device 2 may be used by a medical diagnostic service consumer.

The remote medical diagnosis system 100 may include a plurality of terminals 3. Each terminal 3 may freely select the connection and disconnection to the remote medical diagnosis system 100. Each terminal 3 may be, for example, one used by different individual read-out expert 130. A certain terminal 33 may be used by a plurality of read-out experts 133 and 134.

The service device 1 may be a computing device including a processing unit 11 and a communication unit 12. For example, the service device 1 may be a server.

The processing unit 11 can access a storage unit 13 including the reference medical information and the reference readout result.

In one embodiment, the storage unit 13 is a peripheral device included in the service device 1, and can be powered by a first power source that supplies power to the communication unit 12 and the processing unit 11.

The service device 1 may be a local network or a metropolitan area network (MAN), a local area network (LAN), or a local area network (LAN) The user can access the storage unit 13 through various wired/wireless communication networks such as the Internet.

The requesting device 2 may be a computing device including a processing unit 211 and a communication unit 212. For example, the requesting device 2 may be a server.

The processing unit 211 can access a storage unit 213 including the reference medical information and the reference readout result.

In one embodiment, the storage unit 213 is a peripheral device included in the requesting device 2, and can be powered by a first power source that supplies power to the communication unit 212 and the processing unit 211.

In another embodiment, the storage unit 213 may be a device provided separately from the requesting device 2. At this time, the requesting device 2 can access the storage unit 313 through a wired/wireless communication network such as a local network, a metropolitan area network (MAN), a local area network (LAN).

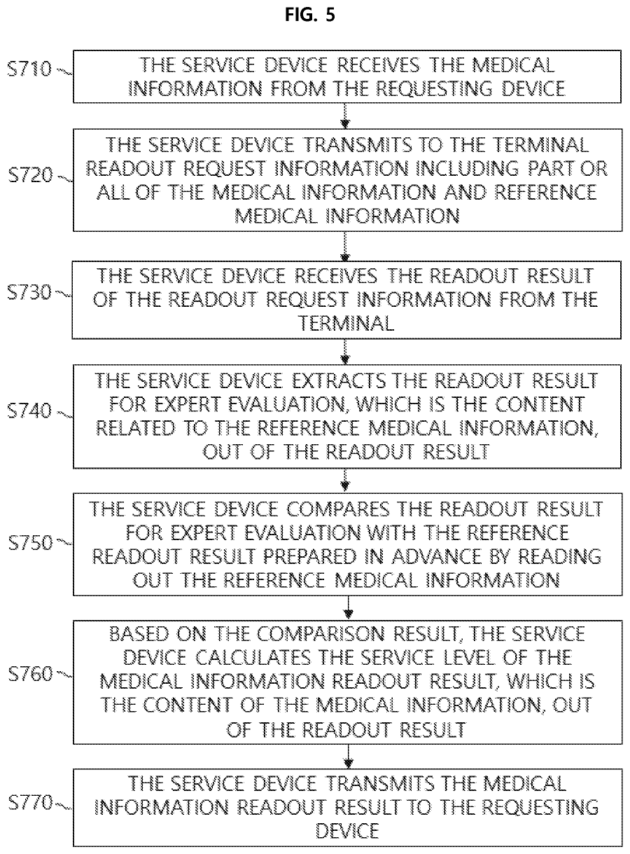

FIG. 5 is a flow diagram illustrating a method of remote medical diagnostic service provided in accordance with an embodiment of the present invention.

In step S710, the service device 1 may receive the medical information from the requesting device 2.

In step S720, the service device 1 may transmit to the terminal 3 readout request information including part or all of the medical information and reference medical information.

In step S730, the service device 1 may receive the readout result of the readout request information from the terminal 3.

In step S740, the service device 1 can extract the readout result for expert evaluation, which is the content related to the reference medical information, out of the readout result.

In step S750, the service device 1 can compare the readout result for expert evaluation with the reference readout result prepared in advance by reading out the reference medical information.

In step S760, based on the comparison result, the service device 1 can calculate the service level of the medical information readout result, which is the content of the medical information, out of the readout result.

In step S770, the service device 1 may transmit the medical information readout result to the requesting device 2.

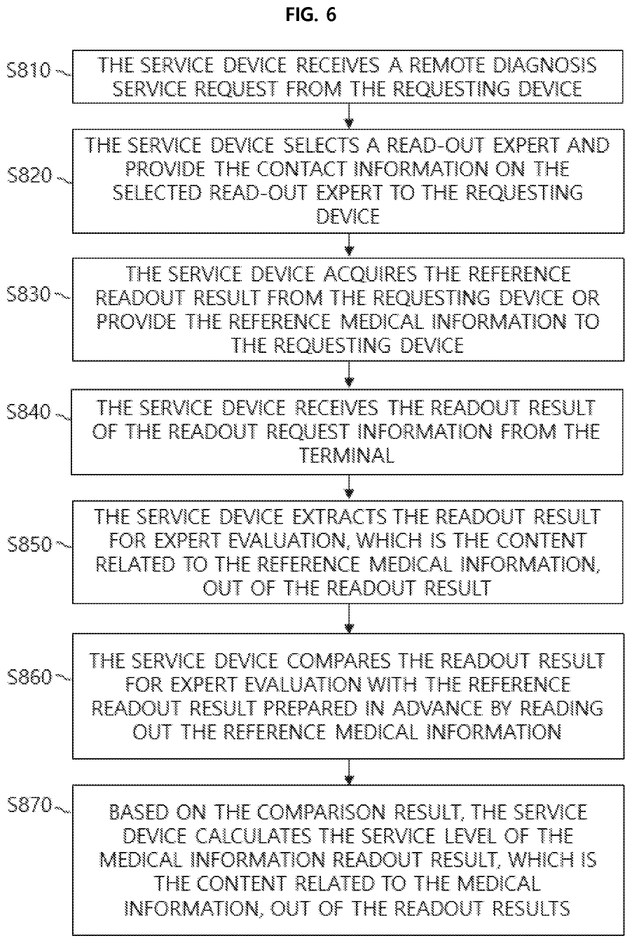

FIG. 6 is a flow chart illustrating a method of remote medical diagnostic service provided in accordance with another embodiment of the present invention.

In step S810, the service device 1 may receive a remote diagnosis service request from the requesting device 2.

In step S820, the service device 1 can select a read-out expert and provide the contact information on the selected read-out expert to the requesting device 2.

In step S830, the service device 1 may acquire the reference readout result from the requesting device 2 or provide the reference medical information to the requesting device.

In step S840, the service device 1 may receive the readout result of the readout request information from the terminal 3.

In step S850, the service device 1 can extract the readout result for expert evaluation, which is the content related to the reference medical information, out of the readout result.

In step S860, the service device 1 may compare the readout result for expert evaluation with the reference readout result prepared in advance by reading out the reference medical information.

In step S870, based on the comparison result, the service device 1 can calculate the service level of the medical information readout result, which is the content related to the medical information, out of the readout results.

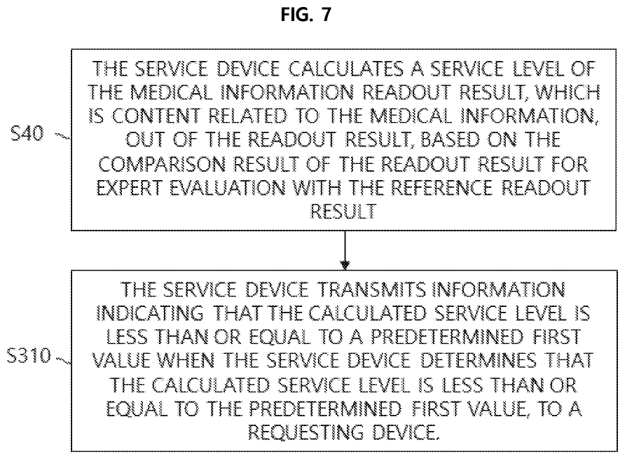

FIG. 7 is a flowchart illustrating a method of utilizing the calculated service level according to an embodiment of the present invention.

The method shown in FIG. 7 may be based on step S40 shown in FIG. 1.

In step S40, the service device may calculate a service level of the medical information readout result, which is content related to the medical information, out of the readout result, based on the comparison result of the readout result for expert evaluation with the reference readout result.

In step S310, the service device may transmit information indicating that the calculated service level is less than or equal to a predetermined first value when the service device determines that the calculated service level is less than or equal to the predetermined first value, to a requesting device.

Here, the requesting device may be a requesting device used by the customer who requested for an external read-out expert to readout the medical information.

According to the method according to FIG. 7, it can be understood that the present invention can be practically used in an application field that informs a customer of the reliability of the medical information readout result which is the result of an external read-out expert reading out the medical information requested by the customer.

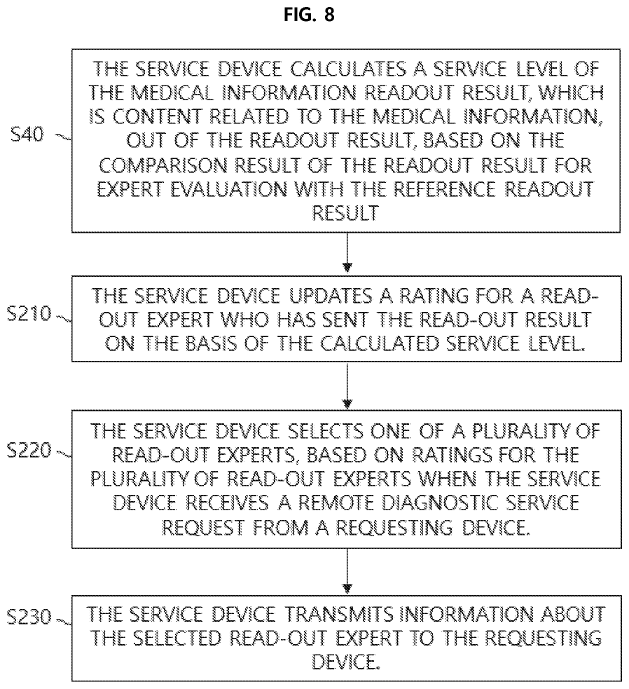

FIG. 8 is a flowchart illustrating a method of utilizing the calculated service level according to another embodiment of the present invention.

The method shown in FIG. 8 may be based on step S40 shown in FIG. 1.

In step S40, the service device may calculate a service level of the medical information readout result, which is content related to the medical information, out of the readout result, based on the comparison result of the readout result for expert evaluation with the reference readout result.

In step S210, the service device may update a rating for a read-out expert who has sent the read-out result on the basis of the calculated service level.

In step S220, the service device may select one of a plurality of read-out experts, based on ratings for the plurality of read-out experts when the service device receives a remote diagnostic service request from a requesting device.

In step S230, the service device may transmit information about the selected read-out expert to the requesting device.

For example, step S230 may be performed between step S11 and step S12 in the embodiment shown in FIG. 1. Or, step S230 may be performed in step S60 in the embodiment shown in FIG. 1. Or, step S230 may be performed in step S111 in the embodiment shown in FIG. 3. However, step S230 is not limited to be conducted at the above-described time point but may be executed at any time point.

At this time, the information about the selected read-out expert may include at least one of the technical field of the selected read-out expert, the rating of the selected read-out expert, the average readout time of the selected read-out expert, and the contact information of the selected read-out expert. However, the information about the selected read-out expert is not limited to those illustrated above.

According to the method according to FIG. 8, it can be appreciated that the present invention can be practically used in applications where an external read-out expert suitable for a remote medical diagnostic service is selected to recommend the selected read-out expert to a remote medical diagnostic service customer.

According to an embodiment of the present invention, a service device including a processing unit and a communication unit adapted to execute at least one of the above-described methods can be provided.

According to one embodiment of the present invention, a non-transitory computer readable storage medium storing one or more programs including instructions executable by an electronic device may be provided. At this time, when the instructions are executed by the electronic device, the electronic device may be adapted to execute at least one of the methods described above. The electronic device may be the service device described above.

The term of medical information described in the present specification is a term for collectively referring to information including at least one of image information and sound information detected from a living organism or a dead organism. The present invention can be applied to an industrial field for diagnosing the condition of a patient, an industrial field for diagnosing the state of living animals, and an industrial field for diagnosing the condition of dead organisms.

While exemplary embodiments in accordance with principles of inventive concepts have been shown, it will be understood that various changes in form and details may be made therein without departing from the spirit and scope of the following claims.

What is claimed is:

1. A method for a remote medical diagnostic service comprising:
a step of receiving from a terminal, by a service device, a readout result of readout request information including a part or all of medical information and reference medical information;
a step of extracting, by the service device, a readout result for expert evaluation, which is content related to the reference medical information, out of the readout result;
a step of comparing, by the service device, the readout result for expert evaluation with a reference readout result which is obtained in advance by reading out the reference medical information; and
a step of calculating, by the service device, a service level of the medical information readout result, which is content related to the medical information, out of the readout result, based on the comparison result.

2. The method of claim 1, further comprising a step of transmitting to a requesting device information indicating that the calculated service level is less than or equal to a predetermined first value when the service device determines that the calculated service level is less than or equal to the predetermined first value.

3. The method according to claim 1, further comprising:
a step of updating, by the service device, a rating for a read-out expert who has sent the read-out result on the basis of the calculated service level;
a step of selecting, by the service device, one of a plurality of read-out experts, based on ratings for the plurality of read-out experts when the service device receives a remote diagnostic service request from a requesting device; and
a step of transmitting, by the service device, information about the selected read-out expert to the requesting device.

4. The method according to claim 1, further comprising:
before the step of receiving, a first step of receiving, by the service device, the medical information from a requesting device;
before the step of receiving, a second step of transmitting, by the service device, the readout request information to the terminal; and
a third step of transmitting, by the service device, the medical information readout result, to the requesting device.

5. The method according to claim 1, further comprising:
by the service device, changing a setting of the service device so that a possibility of selecting a read-out expert who has sent the readout result is reduced when the calculated service level is determined to be less than or equal to a predetermined value, and changing a setting of the service device so that a possibility of selecting a read-out expert who has sent the readout result is increased when the calculated service level is determined to be equal or higher than a predetermined second value.

6. The method according to claim 1, further comprising:
by the service device, changing a setting of the service device so that the read-out expert who have sent the readout result is excluded from candidates for the readout request of other medical information for a predetermined period when the calculated service level is determined to be less than or equal to a predetermined value.

7. The method according to claim 1, wherein,
the medical information includes at least one of image information detected from a living organism or a dead organism and acoustic information detected from a living organism or from a dead organism, and
the medical information is provided for diagnosing a condition of a living organism or provided for diagnosing a condition of a dead organism.

8. The method according to claim 1, further comprising:
a step of receiving after the third step, by the service device, an evaluation score regarding the medical information readout result from the requesting device or another device, or acquiring, by the service device, an evaluation score through a user interface of the service device; and
a step of determining, by the service device, a rating of the read-out expert based on at least one of the evaluation score and the service level and updating a compensation level for the read-out expert based on the determined rating;
wherein,
the service device is adapted to include in the readout request information identification information identifying a read-out expert who will read the medical information and the readout result is deemed to be derived by a read-out expert identified by the identification information.

9. The method according to claim 1, further comprising:
before the step of receiving, by the service device, a step for receiving a remote diagnosis service request from a requesting device; and
before the step of receiving, by the service device, a step for selecting a read-out expert, and providing the requesting device with contact information of the selected read-out expert;
wherein,
the readout result is received from the terminal not passing though the requesting device or passing through the requesting device, and
the readout request information is transferred from the requesting device to the terminal without passing through the service device.

10. The method according to claim 9, further comprising:
before the step of receiving, by the service device, a step for providing the requesting device with the reference medical information; or
before the step of receiving, by the service device, a step for acquiring the reference readout result from the reqeusting device.

11. A service device comprising a communication unit, and a processing unit, wherein, the processing unit is configured to:
using the communication unit, receive a readout result of readout request information including a part or all of medical information and reference medical information from a terminal;
extract a readout result for expert evaluation, which is content related to the reference medical information, out of the readout result;
compare the readout result for expert evaluation with reference readout result which is obtained by reading out the reference medical information; and
calculate a service level of the medical information readout result, which is content related to the medical information, out of the readout result, based on the comparison result.

12. The service device according to claim 11, wherein, the processing unit is further configured to:
before receiving the readout result, using the communication unit, receive the medical information from a requesting device;
before receiving the readout result, using the communication unit, transmit the readout request information to the terminal; and
using the communication unit, transmit the medical information readout result, to the requesting device.

13. A non-transitory computer readable storage medium storing one or more programs, the one or more programs comprising instructions, which when executed by an electronic device, cause the electronic device to:
receive a readout result of readout request information including a part or all of medical information and reference medical information from a terminal;
extract a readout result for expert evaluation, which is content related to the reference medical information, out of the readout result;
compare the readout result for expert evaluation with reference readout result which is obtained by reading out the reference medical information; and
calculate a service level of the medical information readout result, which is content related to the medical information, out of the readout result, based on the comparison result.

14. A non-transitory computer readable storage medium according to claim 13, wherein,
when the instructions are executed by the electronic device, the instructions further cause the electronic device to:
before receiving the readout result, receive the medical information from a requesting device;
before receiving the readout result, transmit the readout request information to the terminal; and
transmit the medical information readout result, to the requesting device.
